SEQUENCE LISTING (4 sequences)

SEQ ID NO: 1 - DNA, 6450 bp, Homo sapiens
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggagctgcacttgctccgtcgggtcgccggcttcaccggaccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcacttccccctataggaatacaaggggccacacagggaaggcagatcccctagttggccaagacttattttaacttgatacactgcagattcagagtgtcctgaagctctgcctctggctttccggtcatgggttccagttaattcatgcctcccatggacctatggagagcaacaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgagcacagccagacttgctcaggtggccctgcacaggctgcagctacctaggaacattccttgcagaccccgcattgcctttgggggtgccctgggatccctggggtagtccagctcttattcatttcccagcgtggccctggttggaagaagcagctgtcaagttgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtagctgctcgggcttctcttggtatgtcttgtttggaaaagtggatttcattcatttctgattgtccagttaagtgatcaccaaaggactgagaatctgggagggcaaaaaaaaaaaaaaaagtttttatgtgcacttaaatttggggacaattttatgtatctgtgttaaggatatgcttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccaaggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaataaagtcatacagtagctcaaaaggcaaccataattctctttggtgcaagtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcatttttttgcatccaattgtgcctgaacttttaaaatatgtaaatgctgccatgttccaaacccatcgtcagtgtgtgtgtttagagctgtgcaccctagaaacaacatacttgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 2 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 3 - DNA, 1560 bp, Homo sapiens
ggctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtgacgcctggccctgaggtgaactggcccacagaggtcacaagctgaagcgtgaactccagtgtgtcaggagcctgggcttcatctttctgctgtgtggtccctcatttgg

SEQ ID NO: 4 - protein, 477 aa, Homo sapiens
MNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

(19) United States
(12) Patent Application Publication
(10) Pub. No.: US 2010/0247602 A1
(43) Pub. Date: Sep. 30, 2010

(54) METHOD OF REDUCING THE EFFECTS OF CYTOSTATIC DRUGS ON BONE MARROW DERIVED CELLS, AND METHODS OF SCREENING

(76) Inventor: Jean-Francois Tanguay, Montreal (CA)

Correspondence Address: KLARQUIST SPARKMAN, LLP, 121 SW SALMON STREET, SUITE 1600, PORTLAND, OR, 97204, US

(21) Appl. No.: 12/679,238

(22) PCT Filed: Sep. 22, 2008
(86) PCT No.: PCT/CA08/01677

§ 371 Date: May 20, 2010

Related U.S. Application Data
(60) Provisional application No. 60/973,931, filed on Sep. 20, 2007.

(51) Int. Cl. A61K 9/00; A61K 31/565; A61K 31/7105; C12N 5/071; G01N 33/50; A61P 9/10

(52) U.S. Cl. 424/423; 514/182; 514/44 A; 435/325; 435/7.21

(57) ABSTRACT

A method of using an estrogen receptor agonist and antagonist to reduce a toxic effect of a cytostatic drug on bone marrow derived cells in a biological system. The methods comprise contacting the cells with a therapeutically effective amount of an estrogen receptor agonist or antagonist, and contacting the cells with a cytostatic agent, whereby the toxic effect of the cytostatic drug on bone marrow derived cells is reduced. Agonists disclosed include 17-beta-estradiol. Antagonists disclosed include antisense nucleic acids and selective estrogen receptor modulators (SERMs). Furthermore, uses and medicaments comprising estrogen receptor agonists and antagonists are provided, as are screening methods for identifying therapeutic candidates for reducing the effect of cytostatic agents, and methods of using estrogen receptor agonists for increasing the proliferation of CD117⁺ cells in a biological system.

### CROSS REFERENCE TO RELATED APPLICATIONS

This application is a PCT application which claims priority on U.S. provisional application Ser. No. 60/973,931, filed on Sep. 20, 2007. All documents above are incorporated herein in their entirety by reference.

### FIELD OF THE INVENTION

The present invention relates to method of reducing the effects of cytostatic drugs on bone marrow derived cells, and methods of screening.

### BACKGROUND OF THE INVENTION

Restenosis at the site of endoluminal procedures is the most significant limitation of percutaneous coronary intervention (PCI) for coronary artery diseases. This process is primarily caused by a disruption of the anatomic and functional integrity of the endothelium at the site of injury.

Local delivery of pharmacologic agents to the site of coronary intervention has been preferred to systemic therapy for the treatment of restenosis. This approach facilitates anti-restenotic agent uptake in injured arterial tissue and promotes local pharmacodynamic effects while attenuating potential systemic toxic side effects. Drug-eluting stents (DES) loaded with rapamycin and paclitaxel have been commercialized for human percutaneous coronary interventions and their protective effects have been mainly attributed to inhibition of smooth muscle cells (SMCs) proliferation. Nevertheless, it has recently been demonstrated that both agents have unfavorable effects on endothelial cells (EC). Rapamycin inhibits proliferation of progenitor cells (PC) and mature EC while paclitaxel attenuates EC migration and adhesion to the lesion. This may affect the reendothelialization and thus limit the global effectiveness of each DES to reduce restenosis or more importantly lead to late thrombosis in high-risk sub-group and possibly on a long term basis to favour vulnerable plaque destabilization or progression of atherosclerosis.

Considering the importance of the endothelium integrity and functionality to prevent restenosis and to influence the atherosclerotic process, pro-healing methods to accelerate restoration of endothelial integrity and function are of major interest. The present inventors as well as others have already contributed to demonstrate that 17-beta-estradiol (E2) acts as a survival factor for EC; increase endothelial proliferation and nitric oxide release; decrease SMC migration and proliferation; and reduce leukocyte adherence and cellular adhesion molecule expression and in vivo, reduce neointima formation and promote the reendothelialization process. These results strongly support the use of E2 for the prevention and the treatment of vascular diseases such as restenosis and vulnerable plaque. Interestingly, it was recently demonstrated, in a mice model of arterial injury, that acceleration of reendothelialization induced by a systemic treatment with estrogens was partially mediated by the mobilization and incorporation of BM derived endothelial PC to the site of injury. In vitro, E2 acts as a survival factor by protecting EPCs against apoptosis induced by serum deprivation and by decreasing EPC senescence via an increase in telomerase activity.

Vascular healing following procedures such as angioplasty or vascular grafting used in coronary bypasses for instance depends on the balance between proliferation of SMCs and regeneration of endothelium. The implantation of vascular endoprosthesis delivering cytostatic drugs such as rapamycin (RAP) or paclitaxel (PAC) reduces the risks of restenosis by their anti-proliferative effect on SMCs.

More recently, it was found that in order to prevent restenosis, it is also useful to promote the regeneration of the injured endothelium to prevent restenosis. However, these drugs have the drawback of also inhibiting proliferation of endothelial cells.

There is a need for a drug that would increase survival of bone marrow derived cells including endothelial progenitor cells in the presence of cytostatic drugs such as rapamycin and paclitaxel.

The present description refers to a number of documents, the content of which is herein incorporated by reference in their entirety.

### SUMMARY OF THE INVENTION

The present invention found that estradiol advantageously increases the survival of bone marrow derived cells in the presence of cytostatic drugs. This allows for the drug to be administered systemically (i.e., through the bloodstream), as opposed to locally which may considerably simplify the method of treating and preventing restenosis and opens the way to other conditions that could benefit vascular healing such as in saphenous vein graft, organ transplantation, ischemia-reperfusion or vulnerable plaque. In addition, systemic administration combined with local administration can provide a more efficient treatment as well as a delayed treatment which can be advantageous under various conditions.

More specifically, in accordance with an aspect of the present invention, there is provided a method of using an estrogen receptor agonist to reduce the toxic effect of a cytostatic drug on bone marrow derived cells in a biological system, comprising contacting the cells with a therapeutically effective amount of the estrogen receptor agonist, and contacting the cells with a cytostatic agent, whereby the toxic effect of the cytostatic drug on bone marrow derived cells is reduced.

In a specific embodiment, the estrogen receptor agonist is 17-beta-estradiol. In another specific embodiment, the estrogen receptor agonist is an agent that increases the expression of an estrogen receptor alpha. In another specific embodiment, the estrogen receptor agonist is an agent that increases the expression of an estrogen receptor alpha In another specific embodiment, the method further comprises a second estrogen receptor agonist. In another specific embodiment, the method further comprises an agent which reduces the expression of the estrogen receptor beta (e.g., an ERβ antisense, SiRNA or the like). In another specific embodiment, the method further comprises an agent which reduces ERβ activation pathway (e.g., an antagonists such as SERM). In another specific embodiment, the cytostatic drug is paclitaxel. In another specific embodiment, the cytostatic drug is rapamycin. In another specific embodiment, the contacting the cells with the estrogen receptor agonist is performed prior to, in combination with or after contacting the cells with the cytostatic drug. In another specific embodiment, the contacting the cells with the estrogen receptor agonist is performed prior to contacting the cells with the cytostatic drug. In another specific embodiment, the bone marrow derived cells are endothelial progenitor cells. In another specific embodiment, the bone marrow derived cells include CD117+ and CD44⁺ cells. In another specific embodiment, the biological system is a mammalian subject. In another specific embodiment, the subject is a human. In another specific embodiment, the subject is suffering or is likely to suffer from a vascular injury caused by: a) saphenous vein graft; b) organ transplantation; c) ischemic-reperfusion; d) vulnerable plaque; e) angioplasty; f) vascular surgery; g) cardiac surgery; h) interventional radiology; i) an infection; j) atherosclerosis; k) high risk plaque; l) interventional cardiology; m) stenosis; or n) restenosis. In another specific embodiment, the contacting is performed through a delivery of the estrogen receptor agonist in the lumen of a blood vessel. In another specific embodiment the contacting is performed through a systemic administration (i.e., through the cardiovascular system) of the estrogen receptor agonist. In another specific embodiment, the systemic administration of the estrogen receptor agonist is by injection. In another specific embodiment, the systemic administration is made through a patch. In another specific embodiment, the delivery is to an injured site of a procedurally traumatized mammalian blood vessel. In another specific embodiment, the delivery is made through the use of a polymer. In another specific embodiment, the delivery is performed with an implantable device. In another specific embodiment, the implantable device is a stent. In another specific embodiment, the implantable device is a graft. In another specific embodiment, the method is performed on an in vitro or ex vivo biological system. In another specific embodiment, the biological system is a cell culture. In another specific embodiment, the biological system is a tissue.

In another specific embodiment, the estrogen receptor alpha is encoded by a nucleic acid sequence comprising SEQ ID NO:1 (GenBank Acc. No. X03635I FIG. 12 A). In another specific embodiment, the estrogen receptor alpha nucleic acid sequence encodes an estrogen receptor alpha polypeptide comprising SEQ ID NO:2 (GenBank Acc. No. X03635; FIG. 12B). In another specific embodiment, the estrogen receptor alpha is encoded by a nucleic acid sequence consisting of SEQ ID NO:1 (GenBank Acc. No. X03635I FIG. 12A). In another specific embodiment, the estrogen receptor alpha nucleic acid sequence encodes an estrogen receptor alpha polypeptide consisting of SEQ ID NO:2 (GenBank Acc. No. X03635; FIG. 12B). In another specific embodiment, the estrogen receptor beta is encoded by a nucleic acid sequence comprising SEQ ID NO:3 (GenBank Acc. NO. X99101; FIG. 13A). In another specific embodiment, the estrogen receptor beta nucleic acid sequence encodes an estrogen receptor alpha polypeptide comprising SEQ ID NO:4 (GenBank Acc. No. X99101; FIG. 13A). In another specific embodiment, the estrogen receptor beta is encoded by a nucleic acid sequence consisting of SEQ ID NO:3 (GenBank Acc. NO. X99101; FIG. 13A). In another specific embodiment, the estrogen receptor beta nucleic acid sequence encodes an estrogen receptor alpha polypeptide consisting of SEQ ID NO:4 (GenBank Acc. No. X99101; FIG. 13A).

In accordance with a further aspect of the present invention, there is provided A method of using an estrogen receptor beta antagonist to reduce the toxic effect of a cytostatic drug on bone marrow derived cells in a biological system, comprising contacting the cells with a therapeutically effective amount of an estrogen receptor beta antagonist, and contacting the cells with a cytostatic agent, whereby the toxic effect of the cytostatic drug on bone marrow derived cells is reduced. In an embodiment, the estrogen receptor beta antagonist is an antisense which reduces the expression of the estrogen receptor beta mRNA. In an embodiment, the estrogen receptor beta antagonist is an agent which reduces estrogen receptor activation pathway. In another embodiment, the estrogen receptor beta antagonist is a selective estrogen receptor down-regulator (SERM).

In accordance with another aspect of the present invention, there is provided a method of screening for therapeutic agents for reducing the effect of a cytostatic agent, comprising contacting the cells with a candidate agent, and contacting the cells with the cytostatic agent, whereby a higher survival of the cells in the presence of the candidate agent than that in the absence thereof is an indication that the agent is able to reduce the effect of the cytostatic agent.

In a specific embodiment, the contacting the cells with the candidate agent is performed prior to contacting the cells with the cytostatic agent.

In accordance with another aspect of the present invention, there is provided a use of a therapeutically effective amount of an estrogen receptor agonist in the manufacture of a medicament for reducing the toxic effect of a cytostatic drug on endothelial progenitor cells. In another specific embodiment, the medicament is in a liquid form suitable for systemic injection through the cardiovascular system.

In accordance with another aspect of the present invention, there is provided a use of a therapeutically effective amount of an estrogen receptor agonist for reducing the toxic effect of a cytostatic drug on bone marrow derived cells.

In accordance with another aspect of the present invention, there is provided a use of a therapeutically effective amount of an estrogen receptor agonist suitable for systemic injection through the cardiovascular system for reducing the toxic effect of a cytostatic drug on bone marrow derived cells.

In accordance with another aspect of the present invention, there is provided a use of a therapeutically effective amount of an estrogen receptor beta antagonist in the manufacture of a medicament for reducing the toxic effect of a cytostatic drug on endothelial progenitor cells. In a specific embodiment, the medicament is in a liquid form suitable for systemic injection through the cardiovascular system.

In accordance with another aspect of the present invention, there is provided a use of a therapeutically effective amount of an estrogen receptor beta antagonist for reducing the toxic effect of a cytostatic drug on bone marrow derived cells.

In accordance with another aspect of the present invention, there is provided a method of using a low concentration of paclitaxel or rapamycin in combination with an estrogen receptor agonist to reduce the mortality or growth inhibition of bone marrow-derived cells (BMDCs) comprising contacting the cell population with an estrogen receptor agonist and paclitaxel or rapamycin, whereby the mortality or growth inhibition of BMDCs is reduced as compared to in the absence thereof and wherein the cell population comprises hematopoietic stem cells, mesenchymal stem cells and stromal cells. In a specific embodiment, said low concentration is below the IC50 concentration. In another specific embodiment, said low concentration is 1/5 of the IC50 concentration. In another specific embodiment, said low concentration is 1/10 of the IC50 concentration. In another embodiment, said low concentration is 1/100 of the IC50 concentration.

### DEFINITIONS

As used herein the terms “bone marrow derived cells” (BMDCs) refer to a cell population derived from bone marrow that includes 1) CD117+ cells including hematopoietic stem cells, endothelial progenitor cells and other progenitor cells, and 2) CD44+ cells including mesenchymal stem cells and stromal cells

As used herein the term “endothelial progenitor cells” (EPCs) refers to bone marrow derived cells that have the ability to differentiate into endothelial cells.

As used herein the term “toxic effect” when used with regards to the effect of cytostatic drugs on bone marrow derived cells refers to, without being so limited, an increase of mortality rate of these cells, a reduction of survival of these cells, a reduction of cell proliferation of these cells, a decrease of differentiation of these cells, and a decrease of mobilization of these cells to sites of injuries, an increase in annexin V positive cells, an increase in the number of apoptotic cells, an increase in necrotic cells.

As used herein the term “cytostatic drug” refers to, without being so limited, to paclitaxel, rapamycin, sirolimus or analogs thereof, zotarolimus, everolimus, tacrolimus, and biolimus.

As used herein the terms “estrogen receptor agonist” refer to estradiol such as 17-beta-estradiol; an estradiol precursor; an active estradiol metabolite such as estrone and estriol; an active analog such as mycoestrogens and phytoestrogens including coumestans, prenylated flavonoid, isoflavones (e.g. genistein, daidzein, biochanin A, formononetin and coumestrol), and lignans; a modulator capable of positively influencing the activity of the estrogen receptor(s) or of enhancing the binding and/or the activity of estradiol towards its receptor such as a selective estrogen receptor modulator (SERM) including tamoxifen and derivative thereof including clomifene, raloxifene, toremifene, bazedoxifene, lasofoxifene, ormeloxifene, tibolone and idoxifene; and an agent which increases the expression of estrogen alpha receptor.

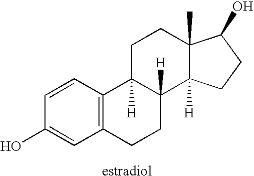

Dehydroepiandrosterone (DHEA) is produced from cholesterol through two cytochrome P450 enzymes. Cholesterol is converted to pregnenolone by the enzyme P450 scc (side chain cleavage) and then another enzyme CYP17A1 converts pregnenolone to 17α-Hydroxypregnenolone and then to DHEA. In humans DHEA is the dominant steroid hormone and precursor of all sex steroids. After side chain cleavage, and either utilizing the delta-5 pathway or the delta-4 pathway, androstenedione becomes another key intermediary. Androstenedione is either converted to testosterone, which in turn undergoes aromatization to estradiol, or, alternatively, androstenedione is aromatized to estrone which is converted to estradiol. As used herein, the terms estradiol precursor include androstenedione and estrone.

As used herein the terms “estrogen receptor beta antagonist” refer to an agent which reduces ERβ activation pathway including an agent which reduces the expression of estrogen receptor beta (i.e., protein or nucleic acid). Non-limiting examples include a selective estrogen receptor down-regulator (SERD) including sulvestrant, ethamoxytriphetol and nafoxidine; and a high dose estradiol such as diethylstilboestrol and ethinyloestradiol; an antisense or siRNA which reduces the expression of the estrogen receptor beta nucleic acid (e.g., mRNA, SEQ ID NO:3); and an antibody binding to the estrogen receptor beta.

As used herein the terms “injured mammalian blood vessel” and “vascular injury” refer both to a procedurally traumatized blood vessel and to a blood vessel affected by an arterial injury that is not the result of a clinical procedure. Without being so limited, these terms include a blood vessel injured by a) saphenous vein graft; b) organ transplantation; c) ischemia-reperfusion; d) vulnerable plaque; e) angioplasty; f) vascular surgery; g) cardiac surgery; h) interventional radiology; i) an infection; j) atherosclerosis; k) high risk plaque l) interventional cardiology; m) stenosis; or n) restenosis.

As used herein the terms “procedurally traumatized mammalian blood vessel” refer to a vessel injured by a surgical/mechanical/cryotherapy/laser intervention into mammalian vasculature. Without being so limited procedural traumas include organ transplantation, such as heart, kidney, liver and the like, e.g., involving vessel anastomosis; vascular surgery, e.g., coronary bypass surgery, biopsy, heart valve replacement, atherectomy, thrombectomy, and the like; transcatheter vascular therapies (TVT) including angioplasty, e.g., laser angioplasty and Percutaneous Transluminal Coronary Angioplasty (PTCA) procedures, employing balloon catheters, and indwelling catheters; vascular grafting using natural or synthetic materials, such as in saphenous vein coronary bypass grafts, dacron and venous grafts used for peripheral arterial reconstruction, etc.; placement of a mechanical shunt, e.g., a PTFE (polytetrafluoroethylene) hemodialysis shunt used for arteriovenous communications; and placement of an intravascular stent, which may be metallic, plastic or a biodegradable polymer.

As used herein the terms “delivery system” includes without being so limited implantable devices, perivascular gels, polymers, microspheres and micelles.

As used herein the terms “implantable device” refers to, without being so limited, stent, shunt, mesh (membrane polymer, intracoronary, endocardiac, epicardiac) and graft made of natural or synthetic materials.

As used herein the terms “injured site” when used to refer to an injured site in a vessel refer to the site of injury or upstream of the injury.

As used herein the terms “biodegradable polymer” refer to a polymer that is biocompatible with 1) target tissues; and 2) the local physiological environment into which the dosage form is to be administered, and capable of being decomposed into biocompatible products by natural biological processes. Such polymers degrade over a period of time preferably between from about 48 hours to about 180 days, preferably from about 1-3 to about 150 days, or from about 3 to about 180 days, or from about 10 to about 30 days. Without being so limited, biodegradable polymers encompassed by the present invention include polylactic acid (PLLA).

As used herein the terms “effective amount of biodegradable polymer” refers to an amount of polymer that enables the loading of as much estrogen receptor agonist as possible in accordance with the present invention. The precise amount of polymer thus depends on its nature and on the nature of the estrogen receptor agonist. Polymers such as PEA (poly(ester amide)) from Medivas™ enables the loading of therapeutic agent in an amount about equal to its own weight (e.g. for 500 μg of polymer, up to 500 μg of estrogen receptor agonist can be loaded). A top coat of polymer can also be applied in addition to this amount to decrease release speed. The present invention also encompasses the chemical coupling of the estrogen receptor agonist to the polymer to slow down its release from this polymer.

As used herein the terms “therapeutically effective amount” refers to an amount sufficient to procure a beneficial effect to the biological system. Any amount of a pharmaceutical composition can be administered to a subject. When implantable devices such as stents are used, amounts of 1 to 5000 μg/kg of subject body weight are typically used to effectively prevents, delays or reduces the toxic effect of cytostatic agents on bone marrow derived cells.

As used herein the term “reduces” in the context of toxic effect refers to any prevention, delay, or decrease of the toxic effect.

As used herein the terms “biological system” refers to a cell or cells, a tissue or a subject.

As used herein the terms “in combination with” in the context of administration (contacting) of at least two therapeutic agents, refers to an administration of at least two agents at the same time in a biological system either separately or together and in particular either in the same delivery system or in different delivery systems.

As used herein the terms “prior to” or “before” in the context of contacting cells (or administration of) with at least two therapeutic agents, refers to a release of a first agent at a time prior to (or overlapping with) the release of the second agent so that the release of the first agent starts before the release of the second agent. The release of the at least two agents can be achieved either in the same delivery system or in different delivery systems. For instance, in the context of a stent used as a delivery system, the stent could have multiple coatings for controlled release enabling the release of the first agent prior to the second agent.

As used herein the terms “controlled release polymer coating” refers to a polymer coating that dispenses the therapeutic agent that it contains in the body gradually. It includes delayed release, fast and slow release.

As used herein the term “subject” is meant to refer to any mammal including human, mice, rat, dog, rabbit, cat, pig, cow, monkey, horse, etc. In a particular embodiment, it refers to a human.

One embodiment of the invention provides a method for biologically stenting a procedurally traumatized mammalian blood vessel. The method comprises administering to the blood vessel an amount of an estrogen receptor agonist in a vehicle effective to biologically stent the vessel. As used herein, “biological stenting” means the fixation of the vascular lumen in a dilated state near its maximal systolic diameter, e.g., the diameter achieved following balloon dilation and maintained by systolic pressure. The method comprises the administration of an effective amount of an estrogen receptor agonist to the blood vessel. Preferably, the estrogen receptor agonist is dispersed in a pharmaceutically acceptable liquid carrier. Preferably, a portion of the amount administered penetrates to at least about 6 to 9 cell layers of the inner tunica media of the vessel and so is effective to biologically stent the vessel but may, as with 17beta-estradiol, penetrate much deeper than that.

The present invention encompasses using cytostatic drugs in amounts higher than those used alone in combination with an estrogen receptor agonist.

The present invention encompasses using in the method of the invention an estrogen receptor agonist alone or in combination with an agent able to reduce activation and/or expression of an estrogen receptor beta (estrogen receptor beta antagonist). In specific embodiments, the agent (i.e., estrogen receptor beta antagonist) is an antisense such as those described in U.S. Pat. No. 7,235,534 to Tanguay et al. In other embodiments, the agent is a small interference (siRNA) or a small hairpin RNA (shRNA). siRNAs and shRNAs have been successfully used to suppress the expression of various genes in the cardiovascular field (see Dev K K, Using RNAi in the clinic. IDrugs. 2006 April; 9(4):279-82; Sugano M. et al., SiRNA targeting SHP-1 accelerates angiogenesis in a rat model of hindlimb ischemia. Atherosclerosis. 2007 March; 191(1):33-9; Takahashi et al., Functional role of stromal interaction molecule 1 (STIM1) in vascular smooth muscle cells. Biochem Biophys Res Commun. 2007 Oct. 5; 361(4):934-40; lantorno M. et al., Ghrelin has novel vascular actions that mimic PI 3-kinase-dependent actions of insulin to stimulate production of NO from endothelial cells. Am J Physiol Endocrinol Metab. 2007 March; 292(3):E756-64; Platt M O et al., Expression of cathepsin K is regulated by shear stress in cultured endothelial cells and is increased in endothelium in human atherosclerosis. Am J Physiol Heart Circ Physiol. 2007 March; 292(3):H1479-86; Cashman S M et al., Inhibition of choroidal neovascularization by adenovirus-mediated delivery of short hairpin RNAs targeting VEGF as a potential therapy for AMD. Invest Opthalmol Vis Sci. 2006 August; 47(8):3496-504; Hecke A. et al., Successful silencing of plasminogen activator inhibitor-1 in human vascular endothelial cells using small interfering RNA. Thromb Haemost. 2006 May; 95(5):857-64).

Generally, the principle behind antisense technology is that an antisense molecule hybridizes to a target nucleic acid and effects modulation of gene expression such as transcription, splicing, translocation of the RNA to the site of protein translation, translation of protein from the RNA. The modulation of gene expression can be achieved by, for example, target degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi is a form of antisense-mediated gene silencing involving the introduction of dsRNA (typically of less than 30 nucleotides in length, and generally about 19 to 24 nucleotides in length) leading to the sequence-specific reduction of targeted endogenous mRNA levels, here the RNA transcript of the estrogen receptor beta gene (e.g., SEQ ID NO:3, GenBank Accession No. X99101). Such dsRNA are generally substantially complementary to at least part of an RNA transcript of the estrogen receptor beta gene gene (GENE ID 2100; NCBI references NC_{—}000014.7; NT_{—}026437.11; AC_{—}000057.1; NW_{—}001838111.1). Another example of modulation of gene expression is the RNA analogue Locked Nucleic Acid (LNA). Other examples relate to double stranded nucleic acid molecules including small nucleic acid molecules, such as short interfering nucleic acid (siNA), short interfering RNA (siRNA), micro-RNA (miRNA). The use of single stranded antisense oligonucleotides (ASO) is also encompassed by the method of the present invention. Sequence-specificity makes antisense compounds extremely attractive as therapeutics to selectively modulate the expression of genes involved in the pathogenesis of any one of a variety of diseases.

Chemically modified nucleosides are routinely used for incorporation into antisense compounds to enhance one or more properties, such as nuclease resistance, pharmacokinetics or affinity for a target RNA.

As used herein “antisense molecule” is meant to refer to an oligomeric molecule, particularly an antisense oligonucleotide for use in modulating the activity or function of nucleic acid molecules encoding an estrogen beta receptor polypeptide (e.g., the polypeptide of SEQ ID NO: 4), ultimately modulating the amount of said estrogen beta receptor in producer cells located in normal distal or surrounding tissues. This is accomplished by providing oligonucleotide molecules which specifically hybridize with one or more nucleic acids encoding estrogen beta receptor (such as SEQ ID NO:3). As used herein, the term “nucleic acid encoding an estrogen beta receptor polypeptide” encompasses DNA encoding said polypeptide, RNA (including pre-mRNA and mRNA) transcribed from such DNA, and also cDNA derived from such RNA (e.g., a nucleic acid comprising the coding sequence of the nucleotide sequence set forth in SEQ ID NO: 3). The specific hybridization of an oligomeric compound with its target nucleic acid interferes with the normal function of the nucleic acid. The overall effect of such interference with target nucleic acid function is modulation of the expression of the estrogen receptor beta. In the context of the present invention, “modulation” means either an increase (stimulation) or a decrease (inhibition) in the expression of a gene.

In the context of this invention, “hybridization” means hydrogen bonding between complementary nucleoside or nucleotide bases. Terms “specifically hybridizable” and “complementary” are the terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. Preferably, the antisense compound is at least 80% complementary, at least 90% complementary; at least 95% complementary or at least 95% complementary to the nucleic acid sequence encoding the estrogen receptor beta polypeptide (SEQ ID NOs: 3 and 4). An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed. Such conditions may comprise, for example, 400 mM NaCl, 40 mM PIPES pH 6.4, 1 mM EDTA, at 50 to 70° C. for 12 to 16 hours, followed by washing. The skilled person will be able to determine the set of conditions most appropriate for a test of complementarity of two sequences in accordance with the ultimate application of the hybridized nucleotides.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. Examples of modified nucleotides include a 2′-O-methyl modified nucleotide, a nucleotide comprising a 5′-phosphorothioate group, a terminal nucleotide linked to a cholesteryl derivative, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate and a non-natural base comprising nucleotide.

Methods to produce antisense molecules directed against a nucleic acid are well known in the art. The antisense molecules of the invention may be synthesized in vitro or in vivo.

The antisense molecule may be expressed from recombinant viral vectors, such as vectors derived from adenoviruses, adeno-associated viruses, retroviruses, herpesviruses, and the like. Such vectors typically comprises a sequence encoding an antisense molecule of interest (e.g., a dsRNA specific for estrogen receptor beta) and a suitable promoter operatively linked to the antisense molecule for expressing the antisense molecule. The vector may also comprise other sequences, such as regulatory sequences, to allow, for example, expression in a specific cell/tissue/organ, or in a particular intracellular environment/compartment. Methods for generating, selecting and using viral vectors are well known in the art.

The present invention comprises using more than one estrogen receptor agonist. In a specific embodiment, the method uses 17-beta-estradiol and an agent that blocks estrogen receptor beta (i.e., estrogen receptor beta antagonist).

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows the proliferation and mortality rate of BMDCs following E2 treatment. Cellular count with trypan blue was performed after one week treatment with different concentrations of E2 diluted in minimal HPGM with 2% FBS. In (A) are the number of living cells for each condition and in (B) represent the percentage of dead cells. Compiled results of 8 mice;

FIG. 2 shows the proliferation of BMDCs. Cellular count after one week treatment with different concentrations of rapamycin or paclitaxel. The IC50 obtained for rapamycin is 10⁻¹⁰M and is 5×10⁻⁹M for paclitaxel. The compiled results of 3 experiments are presented. One-way analysis of variance (ANOVA) followed by Dunnett multiple comparison test. * P<0.01 are significant compared with HPGM;

FIG. 3 shows the mortality rate of drug-treated bone marrow derived cells. Percentage of dead cells in total cell population after one week treatment with different concentrations of rapamycin or paclitaxel diluted in HPGM. The compiled results of 3 experiments for a total of N=6 independent stimulations are presented. One-way analysis of variance (ANOVA) followed by Dunnett multiple comparison test. *: P<0.01 are significant compared with HPGM. LP: P<0.01 compared with E2, ΨΨ: P<0.01 compared with E2 (N=6);

FIG. 4 shows the survival rate of BMDCs following single or combined treatment(s). Percentage of living cells among the total population after a one week treatment with E2 alone (10⁻¹⁰M) or in combination with rapamycin (10⁻¹⁰M) or paclitaxel (5×10⁻⁹M) diluted in HPGM. The survival rate is expressed in percentage of the HPGM used as a reference value. Samples with E2 represent the percentage of live cells when compared to samples treated with the drug alone. N=8 mice. Statistical analyses performed with paired two-tailed t-test. * P<0.0001 compared to HPGM;

FIG. 5 shows the mortality rate of BMDCs following single or combined treatment(s). Percentage of dead cells in total cell population after a 1-week treatment with E2 (10⁻¹⁰ M) alone or in combination with rapamycin (10⁻¹⁰ M) or paclitaxel (5×10⁻⁹ M) diluted in HPGM. Compiled data of 8 mice. Statistical analyses performed with paired two-tailed t-test. * P<0.0001 compared to HPGM alone.

FIG. 6 shows early apoptosis (annexin V), late apoptosis (annexin V+propidium iodine) and necrosis (propidium iodine) levels in BMDCs treated for 48 hours with E2, paclitaxel or rapamycin or the drug only. Each drug was tested at their respective IC50 and IC90 concentration. Statistical analyses performed with paired two-tailed t-test.* P=0.0176 compared to HPGM for the annexin positive cells.

FIG. 7 shows early apoptosis level in BMDCs treated for 48 hours with A) rapamycin or B) paclitaxel alone or in combination with E2 in incomplete medium. Apoptosis level was determined by the percentage of annexin positive cells among gated BMDCS in flow cytometry. N=4 mice. ** P<0.01 and § P<0.05 compared to HPGM alone or with E2 10-9M, *P=0.0176 compared to HPGM alone;

FIG. 8 shows an evaluation by flow cytometry of the differentiation profile of BMDCs. (A) total CD117+ BMDCs; and (B) CD117+ sub-populations following a single or combined treatment with E2 (10⁻⁹M), rapamycin (10⁻¹⁰M) and/or paclitaxel (5×10⁻⁹M) or media alone (HPGM). In combined treatments, BMDCs were pre-treated with E2 for 24 hours before the addition of the drug for a 1-week period;

FIG. 9 shows an evaluation by flow cytometry of the differentiation profile of BMDCs. (A) total CD44+ BMDCs and major sub-populations; and (B) CD44+ small sub-populations following a single or combined treatment with E2 (10⁻⁹M), rapamycin (10⁻¹⁰M) and/or paclitaxel (5×10⁻⁹M), or media alone (HPGM). In combined treatment, BMDCs were pre-treated with E2 for 24 hours before the addition of the drug for a 1-week period;

FIG. 10 shows estradiol's (E2) regulation of ERα expression in mouse bone marrow progenitor cells (mBMPC). (A) Western blot analysis of ERs expression (66 kDa) in mBMPC lysates show that after 24-hour stimulation with various doses of E2, 10-9M was the most effective concentration to up-regulate the expression of ERα. No regulation of ERβ expression was observed (not shown) (B) After 2 weeks in culture including 1 week with various doses of E2, 10-9M was again the optimal dose which increases the ratio of CD117+ progenitor cells among BMDCs. * P<0.05 vs control incomplete media (INC).

FIG. 11 shows the impact of rapamycin and paclitaxel on the expression of ERα and ERβ expressed by BMDCs. After a one week period in culture, BMDCs were treated for 24 hours with a dose ranging from 10-11M to 10-7M of each drug. The expression of ERα and ERβ was evaluated by Western blot using specific antibodies. Results are expressed as the ratio of ERα expression over ERβ. Modifications in the ratio are mainly due to a variation in ERα expression level.

FIG. 12 shows the nucleic acid sequence (A) and protein sequence (B) of the estrogen receptor alpha.

FIG. 13 shows the nucleic acid sequence (A) and protein sequence (B) of the estrogen receptor beta.

### DESCRIPTION OF ILLUSTRATIVE EMBODIMENTS

The applicants have discovered that 17-beta-estradiol (E2) increases survival and decreases mortality of bone marrow derived cells treated with cytostatic drugs thereby contributing to accelerate re-endothelization. The impact of cytostatic drugs such as rapamycin and paclitaxel on cell survival, mortality, on apoptosis and on differentiation was also assayed on bone marrow derived cells isolated from female C57BL/6 mice. It was confirmed that rapamycin and paclitaxel were toxic at low doses (10⁻⁶ to 10⁻¹⁰ M) on bone marrow derived cells.

Bone marrow from female C57BL/6 mice was isolated from long bones of the hind paw and put in culture in fibronectin coated plated with basal media. Two weeks later, the differentiation profile of BM cells in the presence of estradiol, rapamycin, paclitaxel, estradiol/rapamycin or estradiol/paclitaxel were analyzed by flow cytometry using for the expression of stem cells (CD117), stromal/endothelial cells (CD31, CD34, CD90, CD105, CD106, VEGFR2, CD117, CD144), and inflammatory cells (CD45, CD3, CD14) markers. Cells grown in the different conditions were tested for their functionality. Proliferation, sensitivity to apoptosis and ability to form tubules were evaluated by cell based assays. Expression levels of estrogen receptors (ERα and ERβ) were determined by Western blot analyses.

The present invention is illustrated in further details by the following non-limiting examples.

### Example 1

### Material and Methods

### Cell Isolation and Culture

Six-week old C57BL/6 female mice (Jackson Laboratoiries, Bar Harbor, Mass.) were used as bone marrow donors. The mice were euthanized by a ketamine hydrochloride (Bioniche, Belleville, ON) and xylazine (Rompum, Bayer's Inc, Toronto, ON) injections. Total bone marrow cells were collected from femurs and tibia, pooled, and washed twice with phosphate-buffered saline (PBS, Invitrogen corp., Carlsbad, Calif.) contained 2% fetal bovine serum (FBS, Hyclone Laboratories, Logan, Utah). All the bone marrow derived cells (BMDCs) were plated in 6-wells culture plates (Corning, Corning, N.Y.) precoated with rat fibronectin (Calbiochem, San Diego, Calif.), in Hematopoietic Growth Medium (HPGM Lonza, Walkersvelle, Md.) contained 2% fetal bovine serum (FBS, Hyclone Laboratories) and antibiotics (1% penicillin-streptomycin, Invitrogen corp.). Cells were grown in presence of 10 ng/mL platelet derived growth factor (PDGF, Peprotech. Inc, Rocky Hill, N.J.) and 10 ng/mL endothelial growth factor (EGF, Peprotech. Inc.) during the first 7 days. The experimental protocol was approved by the animal care committee of the Montreal Heart Institute.

### Proliferation and Toxicity Assays

BMDCs were cultured and isolated as described above. At day 7, complete culture medium was replaced by an incomplete culture medium: HPGM medium, 2% fetal bovine serum (including EGF and PDGF), and 1% antibiotics (penicillin and streptomycin) with or without E2 (10⁻¹⁰ M to 10⁻⁶ M) (Sigma) and/or rapamycin (10⁻¹¹M to 10⁻⁶ M) (LC Laboratories, Woburn, Mass.) or paclitaxel (10⁻¹¹M to 10⁻⁶ M) (LC laboratories) for another 7 days. In the case of combined treatment, the E2 was added 24 h prior the addition of the drug. First medium change was performed four days after plating and each following three days for a total of 14 days in culture. These culture conditions allowed cohabitation of CD117+ SCs and CD44+ StroCs.

After this treatment, the cells were removed from the 6-well culture plate using a 0.02% solution of (ethylenedinitrilo) tetraacetic acid (EDTA, Sigma) and diluted 1:1 in Trypan blue (Invitrogen corp.) before cell count with a hemacytometer (Hausser Bright-line). Total cells number, white cells and blue stained cells were counted. The proliferation rate was defined as the total number of cells over the number of cells at day 0 of the initiation of the treatment. The survival rate was defined as the number of white cells among the total cell population. The mortality rate was defined as the percentage of blue cells (dead cells) among the total cell population. The IC50 of each drug was determined and all subsequent experiments were achieved with this concentration or as otherwise specified. The combination assay was achieved by a 24-hour pretreatment of BMDCs with different concentrations of E2 (10⁻⁸ to 10⁻¹⁰ M) and the drug was added at its IC50 concentration for a one week treatment. After this time, cells were processed as described above.

### Analysis of the Differentiation Profile

Bone marrow derived cells (BMDCs) can be subdivided into two major populations based on the expression of CD117+. CD117⁺ cells include stem cells mainly dedicated to the hematopoietic branch (HSCs) while CD44⁺ includes mesenchymal and stromal cells (StroCs).

Circulating EPCs were defined by Urbich and Dimmeler as non-endothelial cells (ECs) that show clonal expression and sternness characteristics with the ability of differentiating into ECs. These cells exert multifaceted regulatory roles in the adult vascular system and participate in many physiopathological functions like vascular homeostasis, ischemic tissue vasculogenesis and tumoral angiogenesis. Different populations of circulating EPCs have been identified. They are generally phenotypically and functionally characterized in human by the expression of cell surface markers such as CD133, CD34, and vascular endothelial growth factor receptor 2 (VEGF-R2) and in vitro, by late-outgrowth colony forming unit EC(CFU-EC) formation. In mouse, co-expression of CD117, CD34, VEGFR2 and CD31 is used to define EPC sub-populations and EC.

### Stromal Cells

Within the bone marrow (BM), the stem cell (SC) niche is a specific microenvironment where SCs reside and undergo self-renewal and/or differentiation. Structurally, the BM SC niche is formed by the stromal cells (StroCs), cells which provide physical support and signaling molecules essential to guide stem cells in their function. StroCs include adipocytes, chondrocytes, endothelial cells (ECs), fibroblasts and osteoblasts, and believed to be mainly derived from mesenchymal stem cells (MSC) also found in BM SC niche. This heterogenous population can be identified by the co-expression of various markers such as CD44, CD106, CD105 and CD90. However, the association of CD90 with the stromal phenotype is still controversial.

BMDCs were cultured and isolated as described above. At day 7, complete culture medium was replaced by an incomplete culture medium: HPGM medium, 2% fetal bovine serum (including EGF and PDGF), and 1% antibiotics (penicillin and streptomycin) with or without E2 (10⁻¹⁰ M to 10⁻⁶ M) (Sigma) and/or rapamycin (10⁻¹¹M to 10⁻⁶ M) (LC Laboratories, Woburn, Mass.) or paclitaxel (10⁻¹¹M to 10⁶M) (LC laboratories) for another 7 days. In the case of combined treatment, the E2 was added 24 h prior the addition of the drug. First medium change was performed four days after plating and each following three days for a total of 14 days in culture. These culture conditions allowed cohabitation of CD117+ SCs and CD44+ StroCs.

BMDCs were plated and grown during 7 days before treatment (stimulation). Rapamycin (LC Laboratories) or paclitaxel (LC laboratories) were added for a one week treatment at IC50 concentration. Afterward, cells were washed twice in PBS and non specific binding sites were blocked with 5% normal rat serum (Jackson Immunoresearch Laboratories Inc.). To evaluate the differentiation profile of the BMDCs, quadruple stainings were performed as follow: 1) for the HSC allophycocyanin (APC, Caltag Laboratories, Carlsbad, Calif.) conjugated monoclonal rat-anti-mouse CD117, fluorescein isothiocyanate (FITC, BD pharmingen, San Jose, Calif.) conjugated monoclonal rat anti-mouse CD31 (platelet endothelial cell adhesion molecules-1, PECAM), biotin conjugated monoclonal rat anti-mouse CD34 (Bio, BD pharmingen), growth factor receptor-2 (VEGFR2); and 2) for the stromal cells: APC (Abcam Inc., Cambridge Mass.) conjugated monoclonal rat anti-mouse CD90, FITC conjugated (BD pharmingen) monoclonal rat anti-mouse CD44 (hyaluronic acid receptor), PE conjugated monoclonal rat anti-mouse vascular cell adhesion molecule (V-CAM/CD106, Abcam Inc.), biotin (Abcam Inc.) conjugated monoclonal rat anti-mouse CD105 (Endoglin). Corresponding isotype antibodies were used as negative controls; monoclonal rat anti-mouse IgG2a-APC (Abcam Inc.), monoclonal rat anti-mouse IgG2a-FITC, (Abcam Inc.), monoclonal rat anti-mouse IgG2a-PE (BD pharmingen), monoclonal rat anti-mouse IgG2a-Bio (Abcam Inc.). To analyze the biotin conjugated antibody, a secondary antibody streptavidine-ECD (Beckman coulter, Fullerton, Calif.) was used. Data acquisition was performed using 1×10⁶ events per sample. Of these events, only low-to-medium FSC (forward scattered channel) and SSC (side scattered channel) singlets were gated for analysis of BM subpopulations. In all cases, gated singlets represented 80 to 90% of acquired events. The acquisition and analysis were performed on an Epics Altra cytometer from Beckman using the EXPO™ 32 system ADC software (Beckman Coulter, Fullerton, Calif.).

### Analysis of the Necrosis and Apoptosis Levels

After being cultured and isolated as described above, BMDCs were plated in 12-well culture plate. After one week of culture, rapamycin or paclitaxel were added at their IC 50 and IC90 concentrations. The cells were analyzed 24, 48 and 72 hours later after staining with FITC-conjugated annexin V (Alexis biochemical, San Diego, Calif.) and propidium iodide (PI, Sigma). Acquisition and analysis were performed on an Epics Altra cytometer as described in the section above “Analysis of the differentiation profile”.

### Protein Expression (Western Blot Analyses)

BMDCs were plated in 6-well culture plates and after one week of culture, rapamycin or paclitaxel (10⁻¹¹M to 10⁻⁷ M) was added for 24-hour treatment. Cells were then lyzed and equal amount of total protein (100 ug) was loaded, migrated on 10 or 15% SDS-PAGE (Biorad) gels under reducing conditions, and transblotted onto polyvinylidene difluoride membranes (Millipore, Bedford, Mass.). Membranes were incubated overnight with one of the following antibodies: polyclonal rabbit anti-mouse ERα (Santa Cruz Biotechnology Inc.), polyclonal rabbit anti-mouse ERβ (Alexis biochemical, San Diego, Calif.).

After a one week period in culture, BMDCs were treated for 24 hours with a dose ranging from 10-11M to 10-7M of each drug. The expression of ERα and ERβ was evaluated by Western blot using specific antibodies. Visualization of protein bands was achieved with an anti-rabbit IgG conjugated to horseradish peroxydase (1:20 000 dilution, Santa Cruz Biotechnology Inc.) and a chemoluminescence reagent (Pierce, Rockford, Ill.). Membranes were stripped with Re-Blot Plus™ (International Chemicon) and total protein expression was determined with a goat polyclonal β-actin (1:1000 dilution, Santa Cruz Biotechnology Inc.). Results are presented as the relative expression of the investigated (ERα) protein normalized with the expression of β-actin using digital image densitometry (Biorad).

### Example 2

### Effect of Estradiol on the Number of Living and Dead Bone Marrow Derived Cells

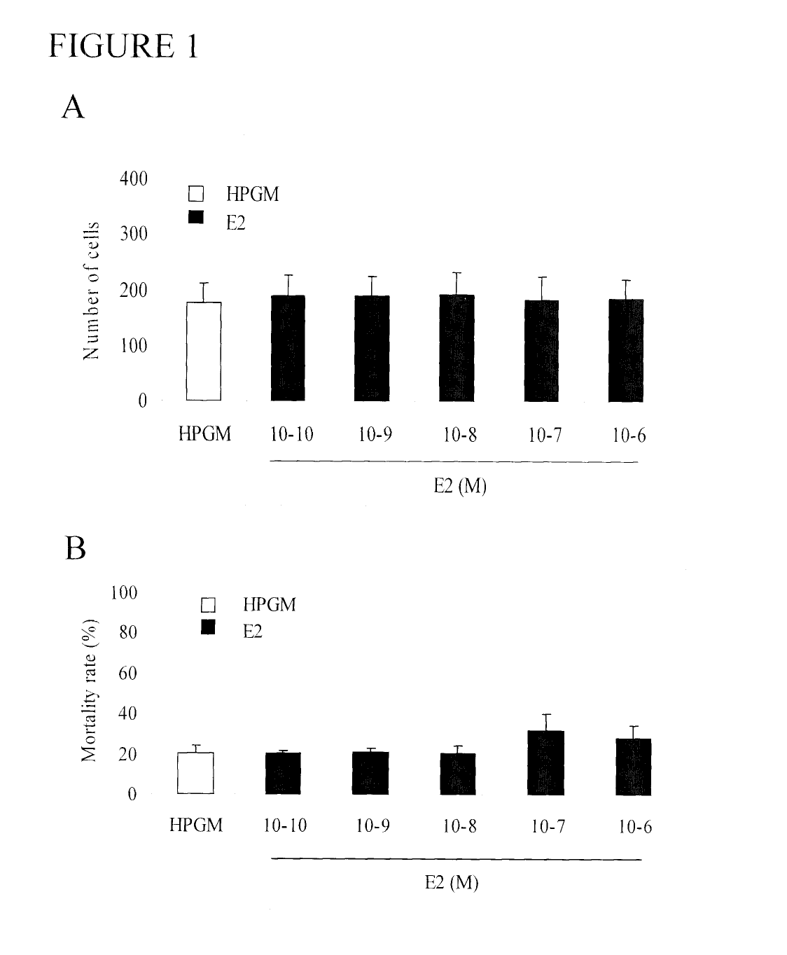

BMDCs were incubated with a dose range of E2 and the total number of living cells and percentage of dead cells were evaluated after a one-week treatment as described in Example 1 above in the section Proliferation and Toxicity assays. The number of living cells (FIG. 1A) and dead cells as evaluated by trypan blue staining and manual count (FIG. 1B) were not affected by the E2 treatments when compared to cells maintained in HPGM.

### Example 3

### Effect of Paclitaxel, or Rapamycin on the Proliferation Rate of Bone Marrow Derived Cells

In order to later evaluate the combined effect of paclitaxel or rapamycin and estradiol the 50% inhibition concentration (IC50) for cell growth for each drug was first determined.

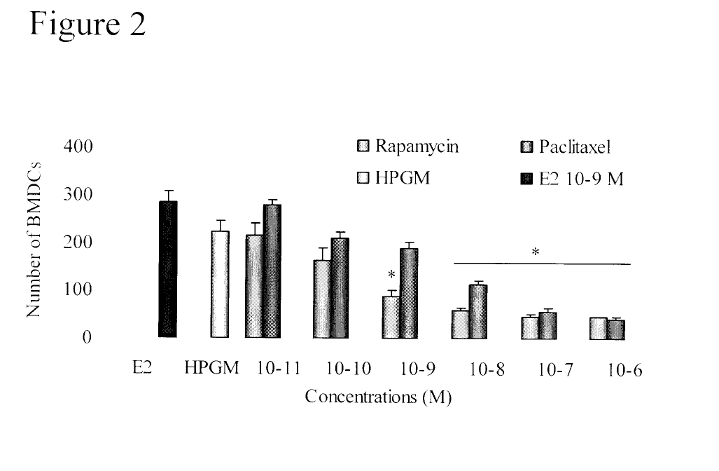

BMDCs (cultured and isolated as described in Example 1 above) were treated during one week with log scale concentration of each drug (FIG. 2). Cultures without drug were used as negative control and cultures with 10⁻⁹ M of E2 were included as reference samples. The IC50 for rapamycin was found to be 10⁻¹⁰ M and with paclitaxel, an IC50 of 5×10⁻⁹ M was found. This value is a balance between the proliferative effect of paclitaxel observed at very low doses and its anti-proliferative effects at higher doses.

### Example 4

### Effect of Paclitaxel or Rapamycin Alone Compared with that of Estradiol on the Mortality Rate of Bone Marrow Derived Cells

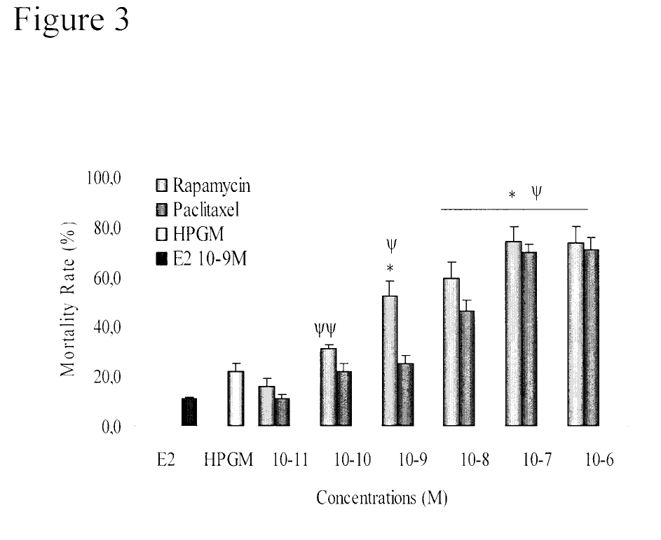

The percentage of dead cells in total cell population after one week treatment with different concentrations of rapamycin or paclitaxel diluted in HPGM was calculated. The compiled results of 3 experiments for a total of N=6 independent stimulations are presented in FIG. 3. One-way analysis of variance (ANOVA) followed by Dunnett multiple comparison test. *: P<0.01 are significant compared with HPGM. Ψ: P<0.01 compared with E2, ΨΨ: P<0.01 compared with E2. (N=6).

The mortality rate was significantly increased in rapamycin treated cultures at doses of 10⁻⁸ M to 10⁻¹⁰ M (FIG. 3). This result indicated that the IC50 was not only a result of growth inhibition but was also linked to a toxicity effect of rapamycin. This trend was not observed with paclitaxel at doses lower than 10⁻⁸ M.

### Example 5

### Effect of Paclitaxel or Rapamycin Alone or in the Presence of Estradiol on the Survival Rate of Bone Marrow Derived Cells

The capacity of E2 to improve the survival rate (percentage of living cells) of BMDCs incubated with rapamycin or paclitaxel was then tested as described in Example 1 above in the section Proliferation and toxicity assays. The percentage of living cells among the total population after a one week treatment with E2 alone (10⁻¹⁰M) or in combination with rapamycin (10⁻¹⁰M) or paclitaxel (5×10⁻⁹M) diluted in HPGM was calculated. The survival rate is expressed in percentage of the HPGM used as a reference value. Samples with E2 represent the percentage of live cells when compared to samples treated with the drug alone. N=8 mice. Statistical analyses performed with paired two-tailed t-test. * P<0.0.0001 compared to HPGM.

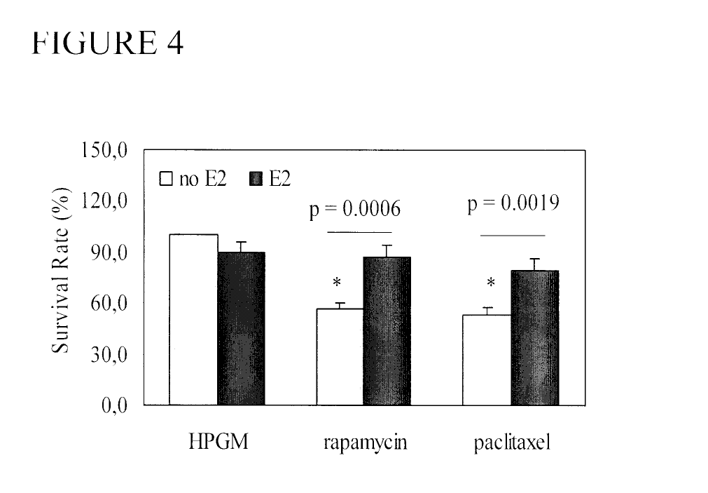

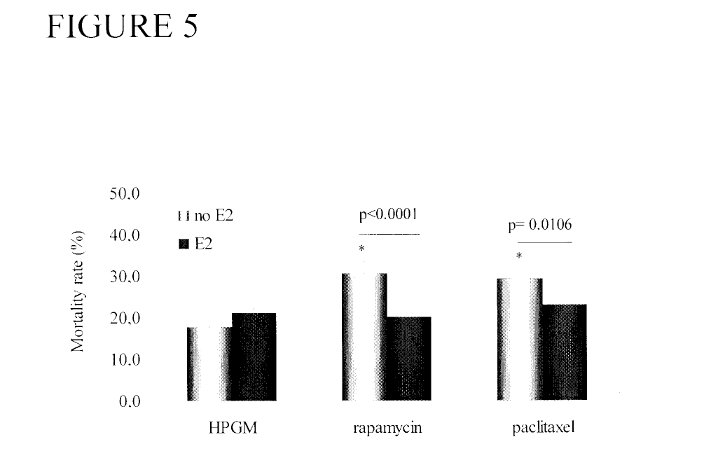

When compared to the cells treated with the drug only, the survival rate was improved by about 30% when 10⁻¹⁰M of E2 was added (FIG. 4). To determine if the increased survival rate was linked to a reduction in cell mortality, combined treatments with drugs at their respective IC50 concentration and E2 at different concentrations (10⁻¹⁰M to 10⁻⁷M) were evaluated. As expected both drugs alone increased by 2-fold the mortality rate when compared with cells maintained in HPGM (P=0.0001) (FIG. 5). When compared with samples treated with the drug alone, the mortality rate was significantly reduced by 7 to 11% when E2 (10⁻¹⁰M) was introduced 24 hours prior the addition of either drug.

### Example 6

### Effect of Paclitaxel, Rapamycin, Alone or in the Presence of Estradiol on the Apoptosis of Bone Marrow Derived Cells

Because cell mortality can occur through necrosis or programmed cell death (apoptosis), a highly regulated pathway, the following study sought to determine which one of these was influenced by E2.

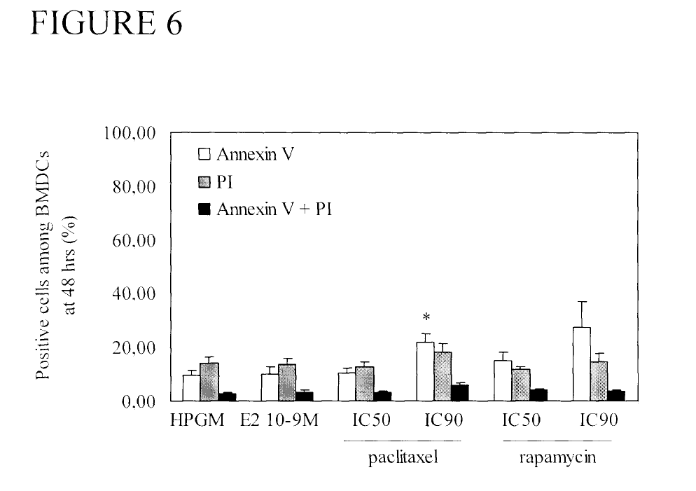

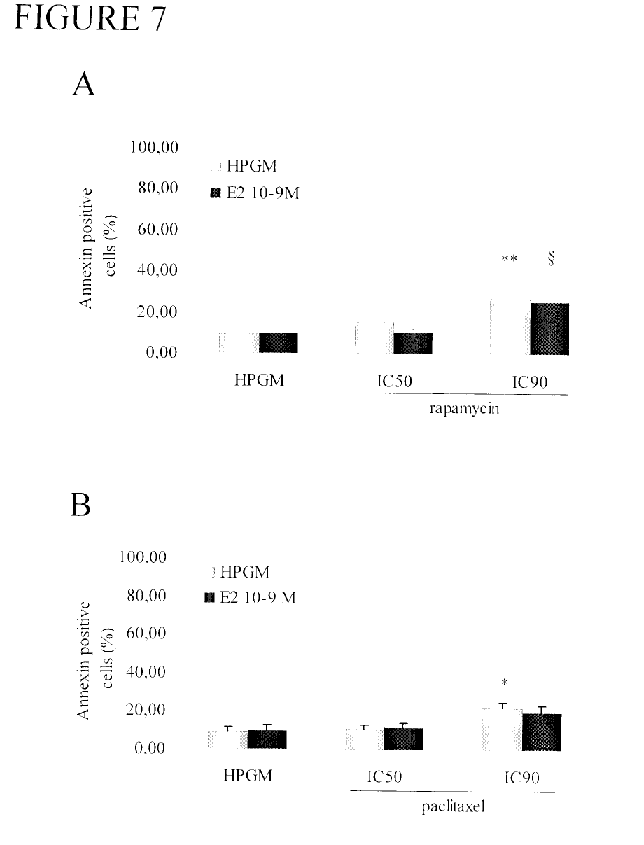

In order to evaluate the effect of E2, paclitaxel or rapamycin on early apoptosis, late apoptosis and necrosis, BMDC were treated with either E2, paclitaxel or rapamycin and stained with annexin V and propodium iodine. Cells positive for annexin V only are considered in the early phases of apoptosis while cells positive for propodium iodine only are in necrosis. Cells which stained for both annexin V and propiodium iodine are in a more advanced apoptotic process (FIG. 6). The effect of E2 on the levels of apoptotic and necrotic cells in rapamycin or paclitaxel-treated cells was evaluated (FIG. 7)

In a first set of experiments, when the BMDCs were incubated during 1 week with E2 or each drug alone or in combination with E2, no appreciable differences in the apoptotic and necrotic cells rates could be detected by flow cytometry. Because annexin V staining is characteristic of early phase apoptosis, the experiments were repeated with cells treated only 24 (data not shown) or 48 hrs with E2, each drug alone or combined with E2 (FIGS. 7A and B).

In this setting, rapamycin and paclitaxel at their respective IC90 doses increased significantly the number of annexin V positive cells, an effect that could not be blocked by E2 in the 48 hrs stimulations (FIGS. 7A and 7B). However, E2 tended to reduce the percentage of annexin V positive cells when compared to rapamycin IC50 alone (FIG. 7B).

### Example 7

### Effect of Estradiol on the Differentiation of CD117+ Bone Marrow Derived Cells Subpopulations Using Markers CD34, VEGFR2 and CD31

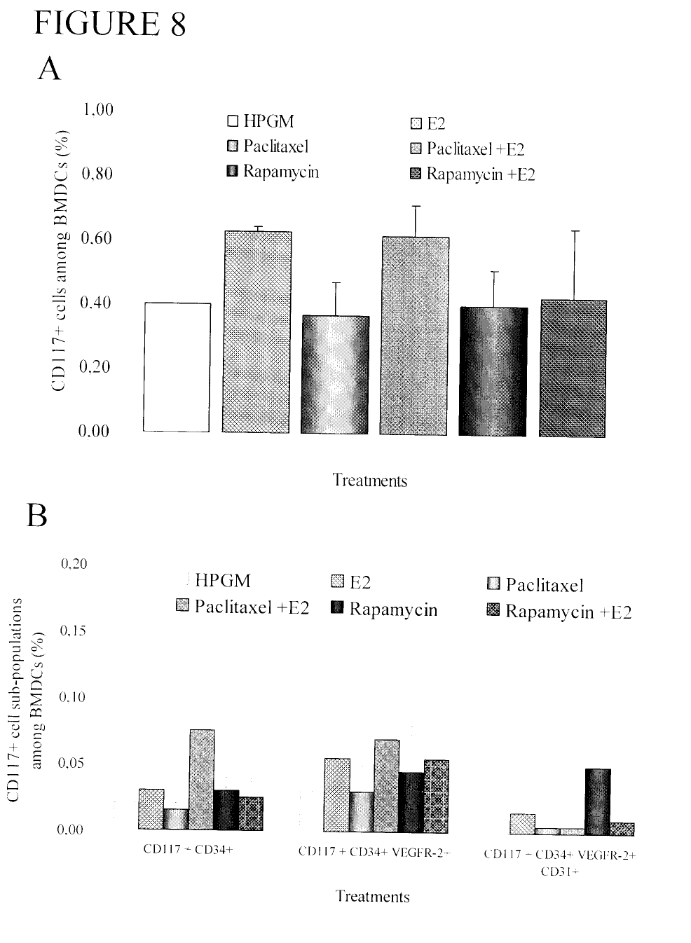

Estradiol was shown to increase the percentage of CD117+ cells in vitro (FIG. 8), the cell population from which EPC arise and which represent less than 0.1% of the total BMDCs. The effect of E2 on subpopulations of CD117+ cells using the following markers was also tested: CD34 found in hematopoietic stem cells, and VEGFR2 and CD31, two endothelial cell markers.

While E2 alone (10⁻⁹M) or in combination with paclitaxel (5×10⁻⁹ M) tended to increase the percentage of CD117+ cells (FIG. 8 A), in the presence of E2, paclitaxel-treated BMDCs comprise a higher level (%) of CD117+ cells. No statistical difference between single or combined treatments was detected among CD117+ sub-populations (FIG. 8).

### Example 8

### Effect of Estradiol on the Differentiation of CD44+ Bone Marrow Derived Cells Subpopulations Using Markers CD106, CD105, and CD90

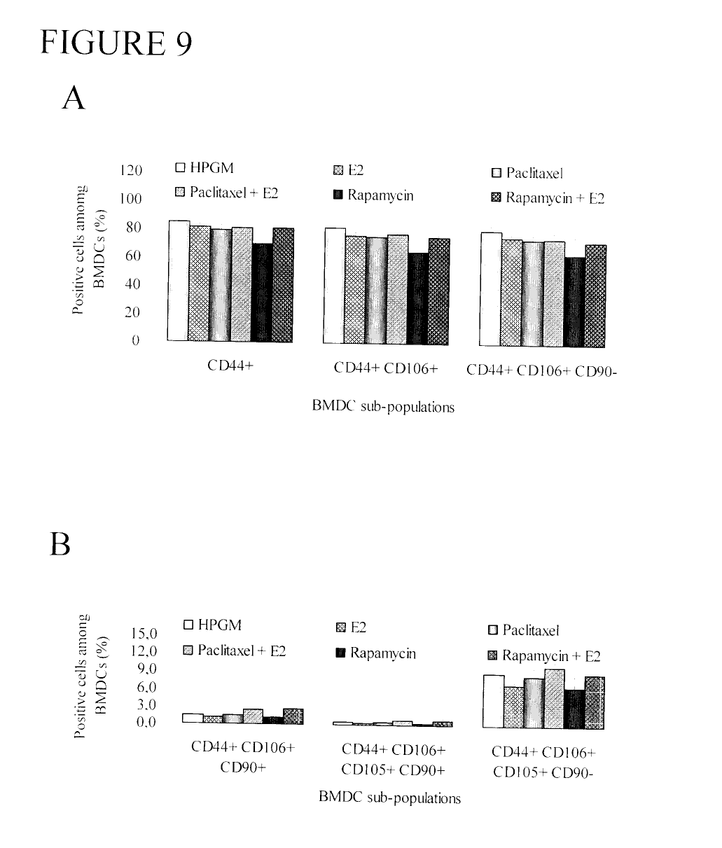

After a one week treatment at the selected concentrations, no effect from E2, paclitaxel (5×10-9M)) or rapamycin (10-10M) alone or in combination was detected on the total or sub-populations of CD44+ cells (FIG. 9).

BMDCs were treated as described in Example 1 in the sections Cell culture and isolation and protein expression. Cytometric analysis of the various markers (i.e. CD117+, CD44+, etc.) of bone marrow derived cells from the hematopoietic and mesenchymal cell line was then performed. Results are presented in FIG. 9. A one-week treatment with either paclitaxel or rapamycin (at their IC50 concentration) does not affect the differentiation profile of CD117+ or CD44+. However, E2 increases the number of CD117+ cells, which translates into a greater potential of generating EPCs.

Because macrophage-like cells (CD14⁺) is another potential source of EPC, the percentage of CD14⁺ was evaluated by flow cytometry after two weeks in culture, including one week in treatment with various doses of E2. From the day of the isolation from the BM to the end of the 2-week culture, the percentage of CD14⁺ increased but stayed low with less than 0.3% of the total cell population (data not shown). These results indicate that these cells do not constitute a significant source of EPCs i.e., that E2 does not influence the number of CD14+ cells.

### Example 9

### Effect of E2, Rapamycin and Paclitaxel on the Expression of Estrogen Receptors Alpha and Beta on BMDCs

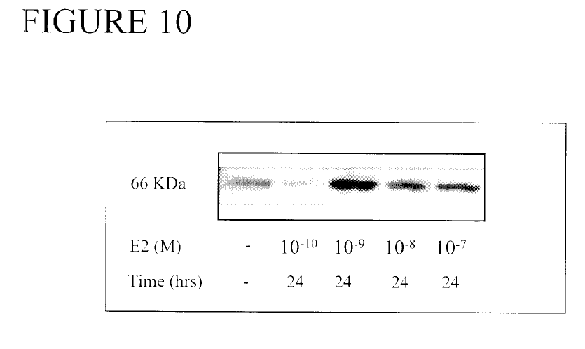

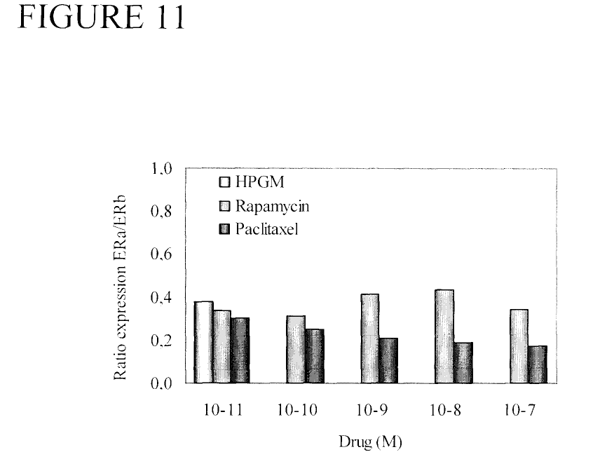

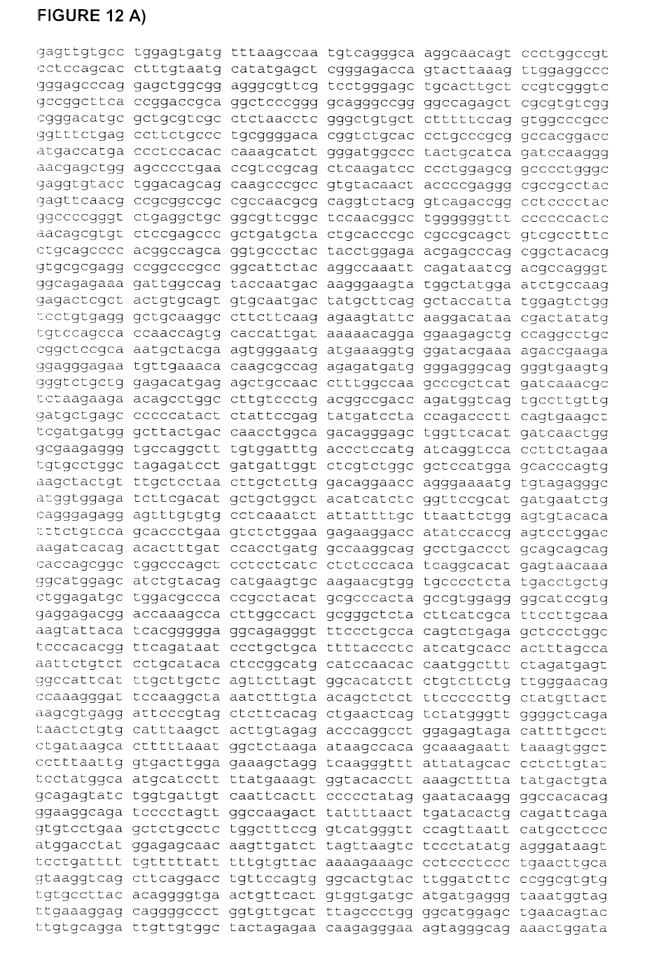

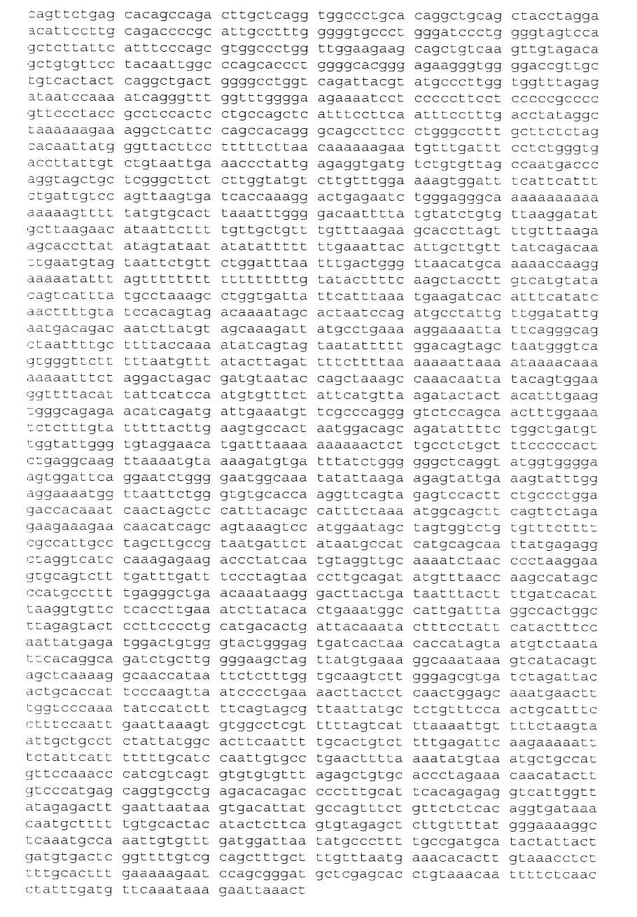

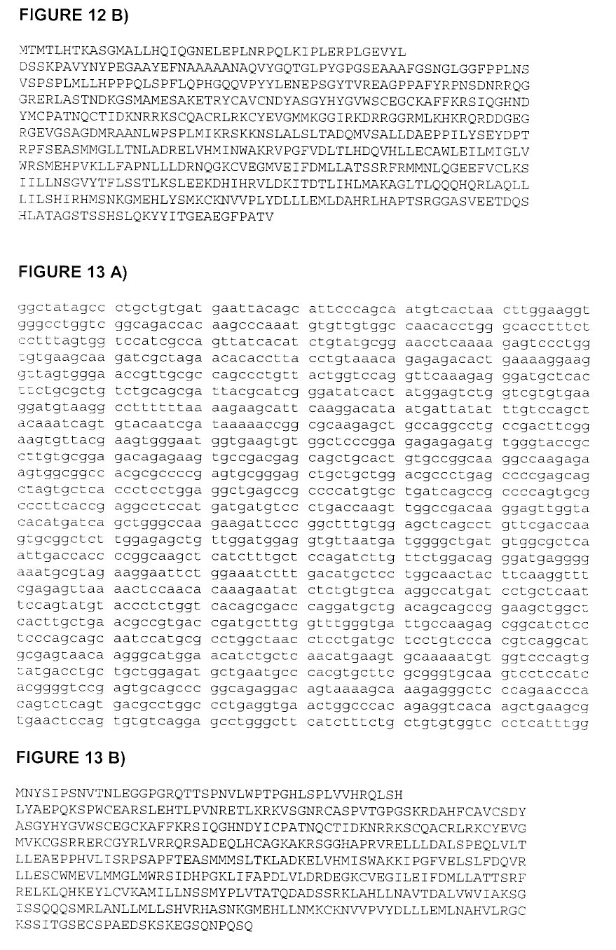

The impact of each of rapamycin or paclitaxel on ERs expressed by BMDCs was tested. Following 24-hour stimulations, ERα and ERβ expression levels were determined by Western blot analysis as described in Example 1 above in the section Protein expression (FIG. 10). When compared to cells maintained in HPGM only, the ERα/ERβ ratio of rapamycin-treated cells remained unchanged. However, with paclitaxel-treated cells, a notable drop in this ratio by up to 50% was observed at 10⁻⁸ and 10⁻⁷M. This change was due to a decrease in ERα expression combined with an induction of ERβ. These results are suspected to be due to the toxic effect of paclitaxel at high doses.

Although the present invention has been described hereinabove by way of specific embodiments thereof, it can be modified, without departing from the spirit and nature of the subject invention as defined in the appended claims.

1. A method of using an estrogen receptor agonist to reduce a toxic effect of a cytostatic drug on bone marrow derived cells in a biological system, comprising contacting the cells with a therapeutically effective amount of the estrogen receptor agonist, and contacting the cells with the cytostatic drug, whereby the toxic effect of the cytostatic drug on bone marrow derived cells is reduced.

2. The method of claim 1, wherein the estrogen receptor agonist is 17-beta-estradiol.

3. The method of claim 1, wherein the estrogen receptor agonist is an agent that increases the expression of an estrogen receptor alpha.

4. The method of claim 3, wherein said estrogen receptor alpha is encoded by a nucleic acid sequence comprising a sequence as set forth in SEQ ID NO:1.

5. The method of claim 1, further comprising contacting the cells with an estrogen receptor beta antagonist.

6. The method of claim 5, wherein said estrogen receptor beta antagonist is an antisense which reduces the expression of mRNA of the estrogen receptor beta.

7. The method of claim 5, wherein said mRNA of the estrogen receptor beta encodes an estrogen receptor beta polypeptide comprising a sequence as set forth in SEQ ID NO:4.

8. The method of claim 6, wherein said mRNA of the estrogen receptor beta comprises a sequence as set forth in SEQ ID NO:3.

9. The method of claim 1, further comprising a second estrogen receptor agonist.

10. The method of claim 1, wherein the cytostatic drug is paclitaxel and/or rapamycin.

11-12. (canceled)

13. The method of claim 1, wherein the contacting the cells with the estrogen receptor agonist is performed prior to contacting the cells with the cytostatic drug.

14. The method of claim 1, wherein the bone marrow derived cells are endothelial progenitor cells.

15. The method of claim 1, wherein the bone marrow derived cells are CD44+ or CD117+ cells.

16. The method of claim 1, wherein the biological system is a mammalian subject.

17. The method of claim 16, wherein the subject is a human.

18. The method of claim 16, wherein said subject suffers or is likely to suffer from a vascular injury caused by: a) saphenous vein graft; b) organ transplantation; c) ischemia-reperfusion; d) vulnerable plaque; e) angioplasty; f) vascular surgery; g) cardiac surgery; h) interventional radiology; i) an infection; j) atherosclerosis; k) high risk plaque; l) interventional cardiology; m) stenosis; or n) restenosis.

19. The method of claim 1, wherein the contacting is performed through a delivery of the estrogen receptor agonist in the lumen of a blood vessel.

20. The method of claim 19, wherein the delivery is (a) to an injured site of a procedurally traumatized mammalian blood vessel; or (b) a systemic administration through the cardiovascular system.

21. The method of claim 20, wherein the delivery is to an injured site of a procedurally traumatized mammalian blood vessel and is performed with an implantable device.

22. (canceled)

23. The method of claim 20, wherein the delivery is a systemic administration through the cardiovascular system and said administration is (a) by injection; or (b) by a patch; or (c) further comprises administration with an implantable device.

24-25. (canceled)

26. The method of claim 21, wherein the implantable device is (a) a stent; or (b) a graft.

27. (canceled)

28. The method of claim 1, which is an in vitro or ex vivo biological system.

29. The method of claim 28, wherein the biological system is (a) a cell culture; or (b) a tissue.

30. (canceled)

31. A method of screening for therapeutic agents for reducing a toxic effect of a cytostatic drug, comprising contacting cells expressing an estrogen receptor with a candidate therapeutic agent, and determining whether said candidate therapeutic agent increases an activity of said estrogen receptor, whereby a higher activity in the presence of the candidate therapeutic agent relative to the absence thereof is an indication that the agent is able to reduce the toxic effect of the cytostatic drug.

32-37. (canceled)

38. A method of increasing the percentage of CD117+ cells in a biological system, comprising contacting the biological system with a therapeutically effective amount of an estrogen receptor agonist.

39. The method of claim 38, wherein said estrogen receptor agonist is 17-beta-estradiol.

40-43. (canceled)

44. The method of claim 1, wherein said toxic effect is (a) an increase in the mortality rate of bone marrow derived cells; (b) a decrease in the proliferation rate of bone marrow derived cells; (c) a decrease in ER alpha expression; (d) a decrease in the ratio of estrogen receptor alpha/estrogen receptor beta expression; (e) an increase in early apoptosis of bone marrow derived cells; and/or (f) an increase in the number of bone marrow derived cells which express annexin V.

45-50. (canceled)

51. The method of claim 44, wherein said estrogen receptor agonist (a) increases the ER alpha expression; and/or decreases said number of bone marrow derived cells which express annexin V.

52. (canceled)

53. A method of using an estrogen receptor beta antagonist to reduce a toxic effect of a cytostatic drug on bone marrow derived cells in a biological system, comprising contacting the cells with a therapeutically effective amount of an estrogen receptor beta antagonist, and contacting the cells with a cytostatic drug, whereby the toxic effect of the cytostatic drug on bone marrow derived cells is reduced.

54. The method of claim 53, wherein said estrogen receptor beta antagonist is (a) an antisense which reduces the expression of mRNA of the estrogen receptor beta; (b) an agent which reduces estrogen receptor activation pathway; and/or (c) a selective estrogen receptor down-regulator (SERM).

55-56. (canceled)

57. A method of using a low concentration of paclitaxel or rapamycin in combination with an estrogen receptor agonist to reduce the mortality or growth inhibition of bone marrow-derived cells (BMDCs) comprising contacting a BMDCs population with an estrogen receptor agonist and paclitaxel or rapamycin, whereby the mortality or growth inhibition of BMDCs is reduced as compared to in the absence thereof and wherein the BMDCs population comprises hematopoietic stem cells, mesenchymal stem cells and stromal cells.
